(19) United States
(12) Patent Application Publication
(10) Pub. No.: US 2026/0321359 A1
(43) Pub. Date: Oct. 15, 2026

(54) EDGE AI CHEMICAL SENSOR FOR AIR, LAND AND SEA APPLICATIONS

(71) Applicant: EyeZense, Inc., Newark, CA (US)

(72) Inventors: Jing Li, Newark, CA (US); Yijiang Lu, Newark, CA (US); Phillip Doan, San Jose, CA (US); Steven Yung, Newark, CA (US); Kejie Bao, Sunnyvale, CA (US)

(21) Appl. No.: 19/643,570

(22) Filed: Apr. 9, 2026

Related U.S. Application Data
(60) Provisional application No. 63/787,506, filed on Apr. 11, 2025.

(51) Int. Cl. G16C 20/20; G01N 27/30; G16C 20/70

(52) U.S. Cl. CPC G16C 20/20; G01N 27/30; G16C 20/70

(57) ABSTRACT

Sensor systems and methods for chemical detection using Edge AI technology combined with micro and nano sensor arrays are described. In some embodiments, the chemical sensors on a carrying vehicle utilize an Edge AI to process the sensor data at the sensor system level and show/display the results for the autonomous response system to take appropriate actions. In some embodiments, the Edge AI includes natural language functions to facilitate the communications with the users/operators.

### CROSS-REFERENCE TO RELATED APPLICATION(S)

This application claims the benefit of U.S. Provisional Patent Application Ser. No. 63/787,506, filed Apr. 11, 2025, and titled “EDGE AI CHEMICAL SENSOR FOR AIR, LAND AND SEA APPLICATIONS,” which is hereby incorporated by reference in its entirety for all purposes.

### FIELD OF THE INVENTION

The present invention relates to the field of AI technology. More specifically, the present invention relates to Edge AI technology with sensors.

### BACKGROUND OF THE INVENTION

In many real-world scenarios, first responders entering hazardous environments must rapidly develop real-time situational awareness to ensure their safety and effectiveness. Although various types of sensors, such as video, acoustic, chemical, and other specialized sensors, are readily available, these sensors are not typically integrated into a cohesive system. Specifically, there is a lack of a unified geospatial 3D map that fuses data from these sensors to provide a comprehensive understanding of the security situation. Furthermore, this critical information is often unavailable to command staff supporting the first responders, limiting their ability to provide timely and informed guidance. As a result, both first responders and command staff face challenges in quickly assessing and responding to hazardous conditions.

### SUMMARY OF THE INVENTION

Sensor systems and methods for chemical detection using Edge AI technology combined with micro and nano sensor arrays are described. In some embodiments, the chemical sensors on a carrying vehicle utilize an Edge AI to process the sensor data at the sensor system level and show/display the results for the autonomous response system to take appropriate actions. In some embodiments, the Edge AI includes natural language functions to facilitate the communications with the users/operators.

### BRIEF DESCRIPTION OF THE DRAWINGS

Embodiments will now be described by way of examples, with reference to the accompanying drawings which are meant to be exemplary and not limiting. For all figures mentioned herein, like numbered elements refer to like elements throughout.

FIG. 1 shows a diagram of a chemical sensor according to some embodiments.

FIG. 2 shows a flowchart of a method of utilizing a chemical sensor according to some embodiments.

FIG. 3 shows a diagram of exemplary chemical sensor chips according to some embodiments.

FIG. 4 shows an exemplary chemical sensor according to some embodiments.

FIG. 5 shows a universal Data Acquisition (DAQ) circuit according to some embodiments.

FIG. 6 shows an exemplary application of oil and pipeline monitoring according to some embodiments.

FIG. 7 shows an exemplary application of fire detection and remediation according to some embodiments.

### DETAILED DESCRIPTION OF THE PREFERRED EMBODIMENT

Sensor systems and methods for chemical detection using Edge Artificial Intelligence (AI) technology combined with micro and nano sensor arrays are described. A system is able to include: an array of sensing elements with sensing materials laid on a pair of electrodes for transforming the chemical signals to electrical signals for measurement, a data acquisition circuit to interpret detection values corresponding to the target chemical types and their concentrations, a firmware to burn in the sensor testing protocol for chemical detection and possible algorithms for data analysis, a data storage memory for sensor data collected by the data acquisition circuit including raw data and processed data, a communication circuit for data transmission through WiFi, Bluetooth®, cellular network, satellite network or a combination thereof to a smartphone, a tablet, a computer and/or a cloud, an app (application) for controlling the sensor system operation and displaying the test results, Edge AI on the chemical sensors to provide accurate predictions/alarms from the test results and trigger the alarm to notify the first responders or the autonomous response systems to take appropriate actions, and chemical sensor fused with video/camera and audio/acoustic sensor to see, hear and smell chemicals for various purposes. In some embodiments, fewer or additional components are implemented.

The Edge AI deployed on a mobile platform (such as a handheld device, drone, Unmanned Aerial Vehicle (UAV), or Unmanned Surveillance Vehicle (USV)—whether underwater, airborne, or ground-based) is capable of performing real-time or remote situational analysis. For instance, the Edge AI is able to assess whether a detected scenario constitutes a hazardous condition, a dangerous event, or a case requiring further investigation or intervention. Based on its analysis, the Edge AI generates a situational assessment signal, which is then transmitted to a remote management center for further action.

As an example, consider an Edge AI sensing system mounted on a vehicle inspecting a section of an oil pipeline or an array of the Edge AI sensing system on the pipeline for stationary detection of a leak. When the system detects a potential chemical anomaly—such as elevated methane and/or hydrogen concentration exceeding a predefined threshold—the Edge AI system processes the sensor data to determine whether a natural gas (methane) or hydrogen leak event is occurring. Upon confirmation, the vehicle transmits either a control command or a treatment recommendation signal to a remote situation management center for appropriate response.

The Edge AI system described herein relates to multiple fields of application including chemical sensing and detection systems. This field covers devices and methods for detecting and analyzing chemical substances, including the use of sensor arrays and signal transformation or Edge AI and Internet of Things (IoT). Since the device incorporates Edge AI technology and communication circuits for data transmission, the device aligns with advancements in IoT and AI-driven sensor systems or micro and nano sensor technology. The use of micro and nano sensor arrays with Edge AI places the system in the domain of advanced sensor technologies.

In some embodiments, the chemical sensors are resistive type of sensors based on the measurement of the resistance of the sensing material before and during the chemical exposures. Chemical sensors are not limited to the resistive type of sensors. The chemical sensors are able to be capacitive sensors, optical sensors, mass sensors, thermal sensors and other types of sensors. The sensing elements are able to be microsensors and/or nanosensors.

In some embodiments, the chemical sensors are in different form factors: body worn, stand alone, handheld, portable and stationary. The chemical sensors are able to be powered by batteries, solar panels and/or wired from the wall.

In some embodiments, the chemical sensors are fused with video/camera and audio/acoustic sensors to combine the see, hear and smell information together for more accurate prediction/alarm.

In some embodiments, the chemical sensors are equipped with temperature sensors and humidity sensors, pressure sensors and altitude sensors for environmental monitoring while performing the chemical detection. This combination is able to provide sophisticated measurements of comprehensive conditions for chemical detection.

In some embodiments, the chemical sensors utilize an Edge AI to process the sensor data at the sensor system level, show/display the results, and generate commands for the autonomous response system to take appropriate actions. In some embodiments, the Edge AI includes natural language functions to facilitate communication with users/operators.

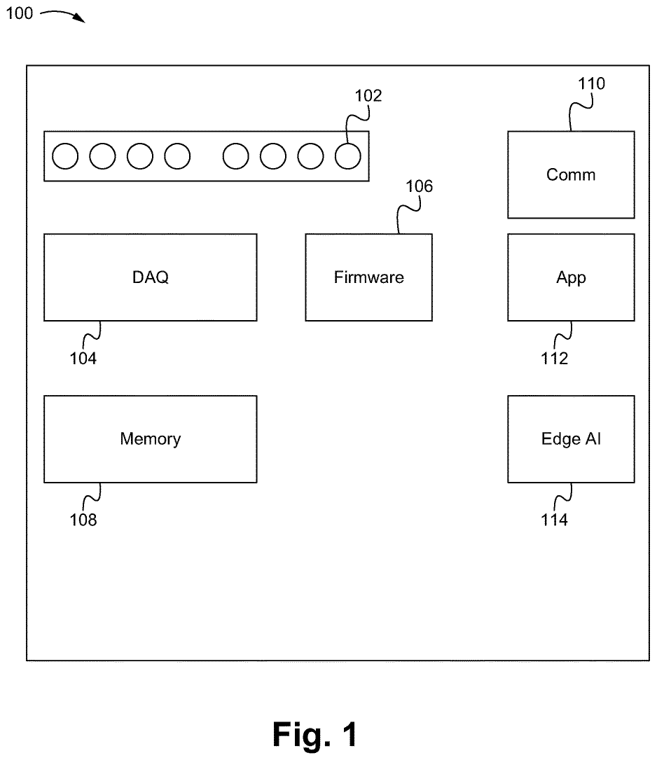

FIG. 1 shows a diagram of a chemical sensor according to some embodiments. The chemical sensor 100 includes an array of sensing elements 102 with sensing materials laid on a pair of electrodes for transforming the chemical signals to electrical signals for measurement. The sensing elements are able to be configured and implemented in any manner.

A Data Acquisition (DAQ) circuit 104 is included to interpret detection values corresponding to the target chemical types and their concentrations.

Firmware 106 is used to burn in the sensor testing protocol for chemical detection and possible algorithms for data analysis. In some embodiments, the firmware is burned into a Memory Protection Unit (MPU) with a series of commands for controlling the sensor DAQ to acquire the sensor data to a flash memory.

A data storage 108 stores sensor data collected by the data acquisition circuit including raw data and processed data. In some embodiments, the data storage 108 is in the MPU and is used for storing the chemical sensor array data. In some embodiments, the data storage 108 is used for AI processing for an event detection.

A communication circuit 110 transmits data through WiFi, Bluetooth®, cellular network, satellite network or a combination thereof to a smartphone, a tablet, a computer and/or a cloud. In some embodiments, the DAQ uses I2C to communicate for data streaming, and the data can be stored on a flash memory on an MPU and then processed using an AI model.

An app (application) 112 controls the sensor system operation and displays the test results. Mobile apps for a phone, tablet or computer are used for controlling the sensor data acquisition, transmission from sensor chip/board to an Edge AI server and then to the cloud, storage on the Edge AI servers and the cloud, and display on the phones, tablets and computers.

Edge AI 114 on the chemical sensor provides accurate predictions/alarms from the test results and triggers the alarm to notify the first responders or the autonomous response systems to take appropriate actions. For example, as soon as the AI model determines an event of interest with sensor fusion data, a notification is generated by the AI model and sent to the autonomous response systems for acting on the event to remediate it.

In some embodiments, the chemical sensor is fused with video/camera and audio/acoustic sensor to see, hear and smell chemicals for various purposes. For example, the Edge AI model for sensor fusion with Audio/Video includes: sensor data pre-processing (e.g., filtering the white noise), sensor data post-processing (e.g., baseline correction), sensor data signal extraction (e.g., algorithm for determining the true sensor signals), set threshold for predicting the event of interest, trigger the alarm to wake up the acoustic sensor for sound detection and the camera to take a snapshot, and all of the see, hear and smell sensor data will be fused using AI model(s) for more accurate predication of an event of interest with minimal or no false alarms, as described in U.S. Ser. No. 19/465,097, filed Jan. 30, 2026, titled, “SYSTEMS FOR AND METHODS OF EDGE AI SENSOR FUSION TO DETECT AND RESPOND TO INCIDENTS OF INTEREST,” which is hereby incorporated by reference in its entirety for all purposes. In some embodiments, the chemical sensor 100 includes fewer or additional components.

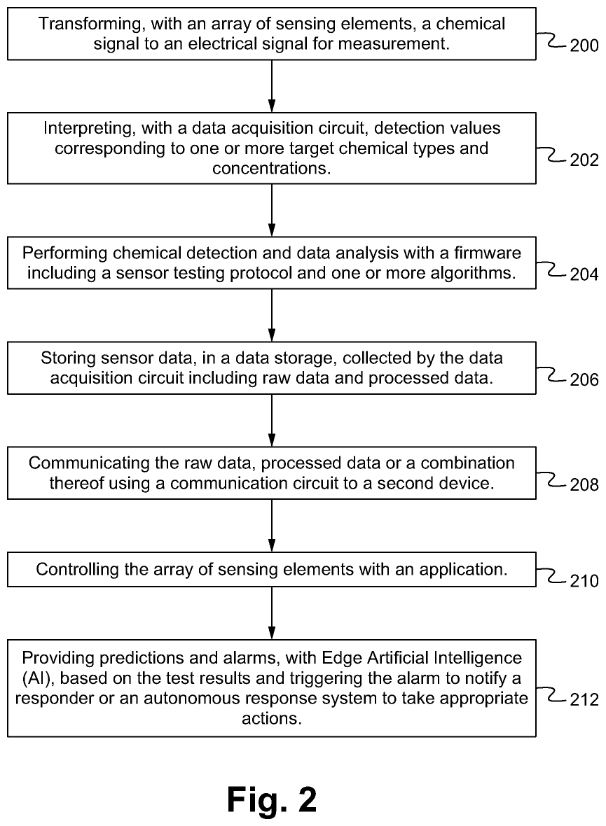

FIG. 2 shows a flowchart of a method of utilizing a chemical sensor according to some embodiments. In the step 200, an array of sensing elements transforms a chemical signal to an electrical signal for measurement. In some embodiments, the array of sensing elements includes sensing materials laid on a pair of electrodes. In some embodiments, the array of sensing elements is selected from resistive, capacitive, optical, mass or thermal sensors. In some embodiments, power is provided to the array of sensing elements with a battery, a solar panel, a wired connection or a combination thereof. In the step 202, a data acquisition circuit interprets detection values corresponding to one or more target chemical types and concentrations. In the step 204, chemical detection and data analysis is performed with a firmware including a sensor testing protocol and one or more algorithms. In the step 206, sensor data is stored in a data storage, collected by the data acquisition circuit including raw data and processed data. In the step 208, the raw data, processed data or a combination thereof is communicated using a communication circuit to a second device. Communicating to second device is through WiFi, Bluetooth®, a satellite network, or a combination thereof. The second device is a smartphone, a tablet, a computer, a cloud device, an Edge AI server or a combination thereof. In the step 210, an application controls the array of sensing elements. In the step 212, Edge Artificial Intelligence (AI) provides predictions and alarms based on the test results and triggering the alarm to notify a responder or an autonomous response system to take appropriate actions. In some embodiments, the order of the steps is modified. In some embodiments, fewer or additional steps are implemented. For example, one or more images are acquired with a camera. In another example, acoustic information is detected with an acoustics sensor. In another example, additional information is acquired with a temperature sensor, a humidity sensor, a pressure sensor, an altitude sensor, or a combination thereof.

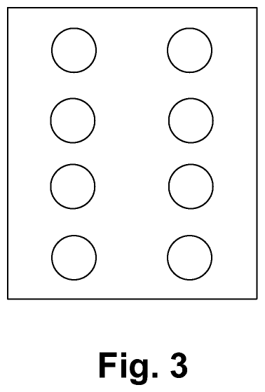

FIG. 3 shows a diagram of exemplary chemical sensor chips according to some embodiments. An n-channel (e.g., 8-channel) sensor array chip is implemented. In some embodiments, each channel can use unique sensing material for complex chemical measurements. In some embodiments, the same material is on multiple channels for redundancy to achieve a high signal to noise ratio.

Using nanostructure sensing materials offers many advantages such as a uniform structure for stable and consistent measurement of chemicals/gases, molecular structure of the materials for ultrasensitive measurement, low power consumption of μW-mW per sensor, small size of the sensor and sensor array for easy deployment, and a multi-channel sensor array for high accuracy measurement using AI models.

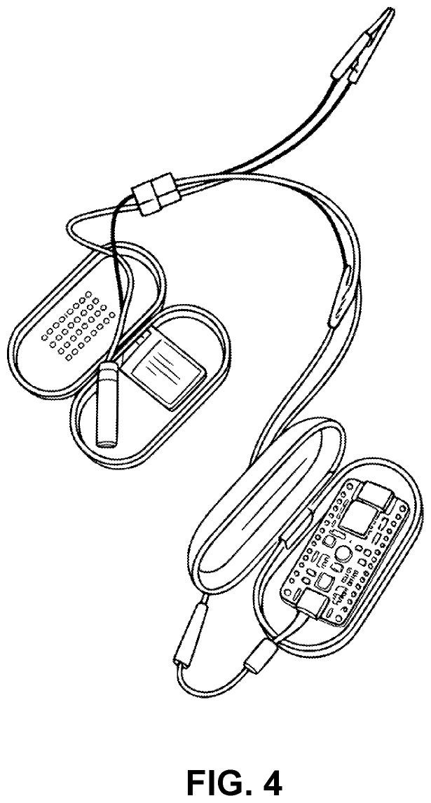

FIG. 4 shows an exemplary chemical sensor according to some embodiments. A chemical sensor chip is contained in a rugged enclosure to protect from the elements.

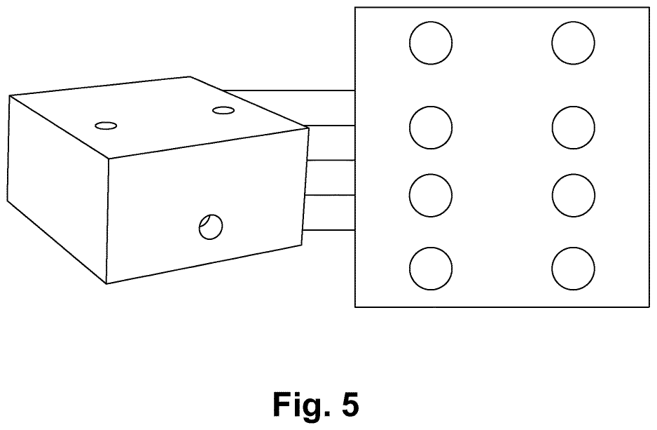

FIG. 5 shows a universal Data Acquisition (DAQ) circuit according to some embodiments. The universal DAQ is flexibly configured to measure the microsensors and nanosensors of the n-channel sensor array in a small footprint.

Since the chemical sensors output analog electrical signals (e.g., voltage, current, resistance), a DAQ is utilized to read the raw signals. Depending on the implementation, the DAQ is configured to amplify a signal, filter noise, and other actions. The DAQ uses an Analog-to-Digital Converter (ADC) to turn the signal into digital values for further processing. In some embodiments, the DAQ records when a measurement was taken and a value of the signal.

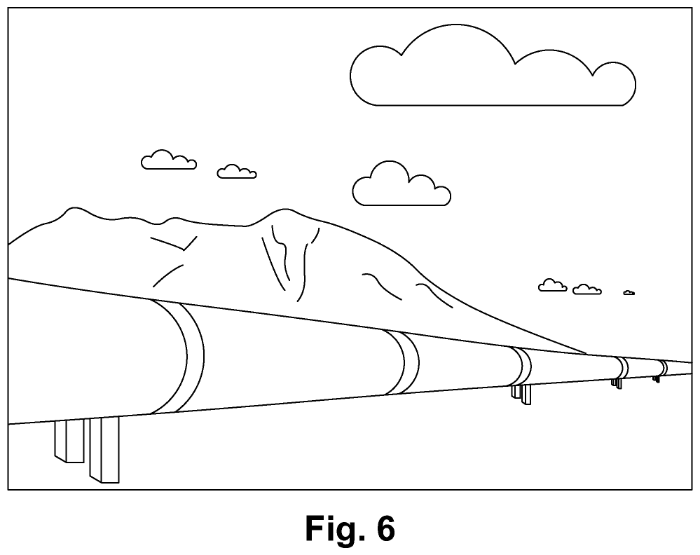

FIG. 6 shows an exemplary application of oil and pipeline monitoring according to some embodiments. The oil pipeline system is able to employ the chemical sensors in one or more edge AI sensors. For example, a chemical sensor is able to be incorporated on/in an oil pipeline. The chemical sensor is able to perform any form of detection. For example, the chemical sensor is able to detect a leak (e.g., gas vapors) or smoke and/or any other information to indicate a potential issue.

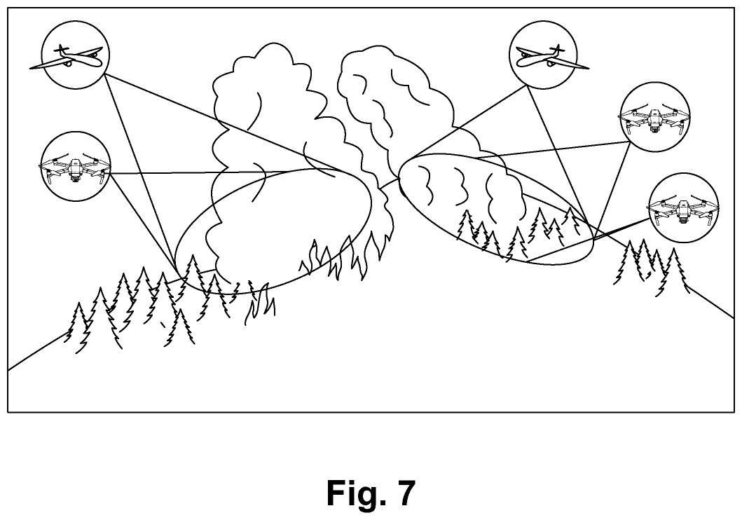

FIG. 7 shows an exemplary application of fire detection and remediation according to some embodiments. The chemical sensors are able to be used to detect smoke and chemicals emitted from a forest fire or fires around a pipeline as a secondary hazard after an attack. By detecting a forest fire, the chemical sensors are able to trigger an alert such that autonomous and non-autonomous vehicles or personnel are able to address the forest fire.

The chemical sensors are able to be utilized in a variety of scenarios. For example, the chemical sensors are used in facilities, infrastructures, pipelines, other stationary locations or non-stationary locations.

To utilize the chemical sensor, the chemical sensor is placed in a location for monitoring (e.g., on an oil or gas pipeline). The chemical sensor detects chemicals of potential issues (e.g., leak). Upon detection of an issue, the chemical sensor communicates information to another device which is able to trigger a response or cause a response to be triggered.

In operation, the chemical sensors are able to be used for 1) oil and gas leak detection, 2) oil and gas efficiency study, 3) fire detection and post fire hazardous detection, 4) home safety alarm—smoke detection, 5) security and safety check at a stadium, mine, closed cargo, airport and other similar situations, 6) battle field for solder's protection from chemical and biological attacks, 7) medical diagnosis, 8) environmental monitoring, 9) green gas detection for global warming, 10) earthquake and volcano irruption detection, or 11) biological and ecological detection of ocean and sea lives. The applications are many more and not limited to these listed above.

The chemical sensor described herein is configured to withstand non-natural hazards and natural hazards/storms (e.g., a windstorm, sandstorm, rainstorm, tornado, hurricane, and others).

The present invention has been described in terms of specific embodiments incorporating details to facilitate the understanding of principles of construction and operation of the invention. Such reference herein to specific embodiments and details thereof is not intended to limit the scope of the claims appended hereto. It is readily apparent to one skilled in the art that other various modifications can be made in the embodiment chosen for illustration without departing from the spirit and scope of the invention as defined by the claims. Features in various examples or embodiments are applicable throughout the Present Specification.

What is claimed is:

1. An apparatus comprising:
an array of sensing elements configured for transforming a chemical signal to an electrical signal for measurement;
a data acquisition circuit configured for interpreting detection values corresponding to one or more target chemical types and concentrations;
a firmware including a sensor testing protocol configured for chemical detection and one or more algorithms for data analysis;
a data storage configured for storing sensor data collected by the data acquisition circuit including raw data and processed data;
a communication circuit configured for data transmission to a second device;
an application configured for controlling the array of sensing elements and displaying test results; and
Edge Artificial Intelligence (AI) configured for providing predictions and alarms from the test results and trigger the alarm to notify a responder or an autonomous response system to take appropriate actions.

2. The apparatus of claim 1 wherein the array of sensing elements includes sensing materials laid on a pair of electrodes.

3. The apparatus of claim 1 wherein the communication circuit for data transmission to the second device is through WiFi, Bluetooth®, a cellular network, a satellite network, or a combination thereof, wherein the second device comprises a smartphone, a tablet, a computer, a cloud device, or a combination thereof.

4. The apparatus of claim 1 wherein the array of sensing elements is selected from resistive, capacitive, optical, mass or thermal sensors.

5. The apparatus of claim 1 wherein the apparatus is configured to be body worn, stand alone, handheld, portable or stationary.

6. The apparatus of claim 1 further comprising a battery, a solar panel, a wired connection, or a combination thereof for providing power to the array of sensing elements.

7. The apparatus of claim 1 further comprising a camera configured for acquiring one or more images.

8. The apparatus of claim 1 further comprising an acoustic sensor configured for detecting acoustic information.

9. The apparatus of claim 1 further comprising a temperature sensor, a humidity sensor, a pressure sensor, an altitude sensor, or a combination thereof.

10. The apparatus of claim 1 wherein the array of sensing elements comprise multiple channels.

11. The apparatus of claim 1 wherein the apparatus of is configured to fuse data received from a second apparatus for redundancy to avoid a false alarm.

12. A method comprising:
transforming, with an array of sensing elements, a chemical signal to an electrical signal for measurement;
interpreting, with a data acquisition circuit, detection values corresponding to one or more target chemical types and concentrations;
performing chemical detection and data analysis with a firmware including a sensor testing protocol and one or more algorithms;
storing sensor data, in a data storage, collected by the data acquisition circuit including raw data and processed data;
communicating the raw data, processed data or a combination thereof using a communication circuit to a second device;
controlling the array of sensing elements with an application; and
providing predictions and alarms, with Edge Artificial Intelligence (AI), based on the test results and triggering the alarm to notify a responder or an autonomous response system to take appropriate actions.

13. The method of claim 12 wherein the array of sensing elements includes sensing materials laid on a pair of electrodes.

14. The method of claim 12 wherein communicating to second device is through WiFi, Bluetooth®, a cellular network, a satellite network, or a combination thereof, wherein the second device comprises a smartphone, a tablet, a computer, a cloud device, or a combination thereof.

15. The method of claim 12 wherein the array of sensing elements is selected from resistive, capacitive, optical, mass or thermal sensors.

16. The method of claim 12 wherein the device is configured to be body worn, stand alone, handheld, portable or stationary.

17. The method of claim 12 further comprising providing power to the array of sensing elements with a battery, a solar panel, a wired connection or a combination thereof.

18. The method of claim 12 further comprising acquiring one or more images with a camera.

19. The method of claim 12 further comprising detecting acoustic information with an acoustic sensor.

20. The method of claim 12 further comprising acquiring additional information with a temperature sensor, a humidity sensor, a pressure sensor, an altitude sensor, or a combination thereof.

21. The method of claim 12 wherein the array of sensing elements comprise multiple channels.

22. The method of claim 12 further comprising fusing data received from a second device for redundancy to avoid a false alarm.

23. A system comprising:
an array of sensing elements configured for transforming a chemical signal to an electrical signal for measurement;
a data acquisition circuit configured for interpreting detection values corresponding to one or more target chemical types and concentrations;
an acoustics sensor configured for detecting audio, a camera configured for acquiring an image, or a combination thereof, wherein the acoustics sensor, the camera or the combination thereof acquire the audio or the image based on the one or more target chemical types and concentrations;
a firmware including a sensor testing protocol configured for chemical detection and one or more algorithms for data analysis;
a data storage configured for storing sensor data collected by the data acquisition circuit including raw data and processed data;
a communication circuit configured for data transmission to a second device;
an application configured for controlling the array of sensing elements and displaying test results; and
Edge Artificial Intelligence (AI) configured for providing predictions and alarms from the test results and trigger the alarm to notify a responder or an autonomous response system to take appropriate actions.

24. The system of claim 23 wherein the array of sensing elements includes sensing materials laid on a pair of electrodes.

25. The system of claim 23 wherein the communication circuit for data transmission to the second device is through WiFi, Bluetooth®, a cellular network, a satellite network, or a combination thereof, wherein the second device comprises a smartphone, a tablet, a computer, a cloud device, or a combination thereof.

26. The system of claim 23 wherein the array of sensing elements is selected from resistive, capacitive, optical, mass or thermal sensors.

27. The system of claim 23 wherein the apparatus is configured to be body worn, stand alone, handheld, portable or stationary.

28. The system of claim 23 further comprising a battery, a solar panel, a wired connection, or a combination thereof for providing power to the array of sensing elements.

29. The system of claim 23 further comprising a temperature sensor, a humidity sensor, a pressure sensor, an altitude sensor, or a combination thereof.

30. The system of claim 23 wherein the array of sensing elements comprise multiple channels.

31. The system of claim 23 wherein the apparatus of is configured to fuse data received from a second apparatus for redundancy to avoid a false alarm.
